(19) United States
(12) Patent Application Publication
(10) Pub. No.: US 2012/0028243 A1
(43) Pub. Date: Feb. 2, 2012

(54) AMPLIFICATION GENIQUE STATISTIQUE POUR L'IDENTIFICATION SANS A PRIORI DE MICRO-ORGANISMES PAR SEQUENCAGE SANS ETAPE DE CLONAGE

(75) Inventors: Christophe Peyrefitte, Aix en Provence (FR); Sebastien Plumet, La Fare les Oliviers (FR)

(73) Assignee: ETAT FRANCAIS represente par LE DELEGUE GENERAL POUR L'ARMEMENT, Arcueil (FR)

(21) Appl. No.: 13/147,008

(22) PCT Filed: Feb. 22, 2010
(86) PCT No.: PCT/FR2010/000148

§ 371 Date: Oct. 13, 2011

(30) Foreign Application Priority Data
Jan. 30, 2009 (FR) 0950575

(51) Int. Cl. C12Q 1/68; C12Q 1/70; C07H 21/04

(52) U.S. Cl. 435/5; 435/6.12; 536/24.33

(57) ABSTRACT

A pair of hexamers and a pair of primers specifically for identifying, without preconceptions, microorganisms in a sample, and a method for identifying one or more microorganisms, and a microorganism identification kit that uses the abovementioned hexamers and primers.

### FIELD OF THE INVENTION

The present invention relates to a pair of hexamers and to a pair of primers, able to anneal sufficiently frequently to a nucleic acid sequence for the amplification of a fragment, such as a 400 nucleotide fragment, of any microorganism genome or transcriptome.

The present invention also relates to a process allowing the generation of DNA fragments directly sequencable, without any preconceived idea regarding the searched species.

The present invention also relates to a microorganism identification kit.

### PROBLEMS AND PRIOR ART

The microbial infections, i.e. infection of a host organism by a microorganism, are generally one of the major morbidity causes within the populations. To establish an efficient diagnosis of a disease or infection and to determine the relevant treatment it is important to quickly and accurately identify the pathogenic agent causing the infection.

Further, in epidemiology, the identification of the microorganism causing the infection is important as it can help determining the source and also the transmission mode of said infection.

Moreover, knowing the microorganism genomic sequence, whole or partial, is the most efficient means for its identification and localisation into the species classification.

Document EP 0 077 149 describes a method, referred as conventional, for identifying unknown microorganisms. This method consists in adding to a sample containing the unknown microorganism, an emitting agent such as a radioactive amino acid, to generate a mixture of emitting products, depending on the microorganism metabolic mechanism. After incubation, the reaction is interrupted and the emitting products are separated, for example by gel plate electrophoresis. The plate can then be radio-autographed by exposing it to a photographic film to obtain thereon an image of the characteristic bands as a means to identify the microorganism. Identification can be done by comparing the means for identifying the unknown microorganism with a set of known identification means, to find a match with one of those known microorganisms. Comparison can be done by deeply examining the unknown identification agents to generate a signal which is compared to signals representing the known identification agents stored in a computer. Alternatively, the emitting products can be detected after separation by deeply examining them directly to provide an identification signal for a computer implemented process.

Document EP 0 151 8855 also describes a conventional method for identifying a microorganism in a sample. The microorganism is submitted to conditions leading to its development in presence of several growth substrates which are individually inoculated with the microorganism. Presence or absence of carbon dioxide, as metabolic by-product of those substrates, is detected by infrared analysis and provides a profile of the unknown microorganism. The identification is performed by comparing this profile with those of known microorganisms processed in the same way.

Also, methods for identifying an unknown microorganism generally include: culturing a sample taken from a diseased patient (blood sample), reculturing on a selective growth medium. Then, the biochemical characterisations of said microorganism are performed, which can be made by: an indole production assay, a gram negative and gram positive bacteria staining, colony morphology study, etc.

These methods require many culturing and are time-consuming especially when the microorganism causing the infection has a slow growth.

On the other hand, detecting a microorganism presence has become important for the diagnosis of diseases.

Sequencing has indeed become an easy process nowadays. It requires obtaining double-stranded DNA in sufficient amount and length, conventionally by using PCR (polymerase chain reaction amplification) or RT-PCR (reverse transcription PCR) on nucleic acid extracted from the studied species.

However, concerning unknown microorganisms, this step is the core issue: RT-PCR is indeed based on hybridization of two DNA primers complementary to the studied sequence and, by definition, it is not possible to design a primer whose sequence is complementary to an unknown sequence.

The solutions described in the art consist of three types:
1) PCR by family: it is based on the use of consensus sequences for some viral families. This solution does not consider a really unknown pathogen, but one suspected to belong to a viral family. Also, all the viral families do not display conserved regions on their genomes.
2) Bioinformatics: it consists of random high-throughput sequencing of small DNA portions (Shotgun) and of a computer-assisted reconstruction of the sequences. However, these processes are long, expensive and require specialized teams.
3) Random PCR: Allander et al. carried out PCR from primers in two parts: an hexamer of 6 random nucleotides (6N, random) which anneals to any DNA sequence, and a tag with a fixed 5′ sequence of 20 nucleotides: 5′ TAG-NNNNNN-3′. This degenerate primer allows the synthesis of a first DNA strand (for instance, reverse transcription), then of a second (with, for instance, a klenow polymerase), initiated randomly. A conventional PCR is then performed with a primer targeting the tags to amplify the generated DNAs. It allows multiplying the DNA strand number, the primers annealing to all the possible sequences. The PCR products are loaded on an agarose gel. As the primers anneal completely randomly, any possible length for the PCR product fragments is obtained, so there are no isolated bands (on the agarose gel we can not see isolated bands but smears). The sequencable PCR products, 300-600 nucleotides in length, are cut from the corresponding piece of gel, then cloned into bacteria after purification. A certain number of clones are then analysed by sequencing and a computer program performs n automatic comparison of the sequences with the genetic databases available on the Internet, such as BLAST. BLAST (basic local alignment search tool) is indeed an algorithm used in bioinformatics for finding similar regions between two or more nucleotide or amino acid sequences. This program allows the significant calculation of similarity percentages between sequences by comparing them to data libraries.

However, having a completely random PCR which generates an infinity of fragments, even when only one nucleic acid, typically a viral genome, is analyzed, can show the following disadvantages:
It is a random amplification of any nucleic acid sequence. Therefore, there is a preparation step of the sample before the molecular biology treatment, then a bioinformatics step to remove any nucleic acids and signal unrelated to the pathogen.
After the reverse PCR (RT-PCR) step, the step of cloning into bacteria is essential. Indeed, a great number of PCR fragments are generated and should be individualised by cloning into bacteria, which increases the number of sequences to make.
A sample containing a pure virus shows exactly the same PCR profile than a sample containing a nucleic acid from the host, from cells, etc. Accordingly, there is no control of the reaction before the ultimate step of sequencing and comparing the sequences, which incurs an over-cost in monopolized work-time and in financial cost.
In the case where one or more viruses could be present in the starting sample, the less represented will be lost during the amplification or analysis of the bacterial clones, “dominated” by the most represented (stochastic effect).
The process is slow and requires many handlings: two or three days are required to achieve the whole reaction, in particular because of the steps of cloning into bacteria, resulting in an impact in terms of human, material and financial resources monopolization.

### SUMMARY OF THE INVENTION

The invention aims to provide a novel method for identifying microorganisms that avoids all or some of the above-mentioned disadvantages.

To this end, the invention relates to a pair of hexamers for PCR for detecting microorganisms, comprising a first hexamer to be positioned at the 3′ end of a first primer and a second hexamer to be positioned at the 3′ end of a second primer, said pair of hexamers being obtainable by the method consisting in:

a) cleaving the genome sequences of at least five viruses from different families into groups of six successive nucleotides,

b) classifying the sequences of six nucleotides, referred as hexamers, according to their occurrence rate,

c) selecting the pairs of hexamers having the most represented occurrence rate, like the first twenty hexamers, preferably the first ten, more preferably, the first five having the higher occurrence rate when compared to the other pairs of hexamers, in order to obtain a pair of primers able to anneal statistically frequently on any nucleic acid matrix.

The present applicant has indeed discovered non degenerate hexamers that, on the contrary, have a fixed sequence. Those hexamers are able to anneal statistically frequently, but not too frequently, in order not to amplify a too high number of sequences for a given source nucleic acid. Therefore, the present applicant selected between the infinite possibilities of existing hexamers.

Appropriately, the first and second hexamers are selected from:

| First hexamer (hexamers | Second hexamer (hexamers |
|---|---|
| written from 5′ to 3′) | written from 5′ to 3′) |
| TTGTAA | TTACAA |
| TCATCA | TTGTAA |
| AAAGAA | TTGTAA |
| GGAAAA | TTTTCC |
| GAAAGA | TCTTTC |
| GGAAGA | TCTTCC |
| AGAAAA | TTTTCT |
| GGGAAA | TTTCCC |
| AAGAAA | TTTCTT |
| AACATG | CATGTT |
| GAAAAA | TTTTTC |
| AAGGAA | TTCCTT |
| GGAAAG | CTTTCC |
| TGGAAA | TTTCCA |
| AAAAAA | TTTTTT |
| GAAGAA | TTCTTC |
| AGGAGA | TCTCCT |
| AAAAAG | CTTTTT |
| AAAAGA | TCTTTT |
| ACATGG | CCATGT |
| AGAAGA | TCTTCT |
| TGATGA | TCATCA |
| TGGAAG | CTTCCA |
| CATGGA | TCCATG |
| AGGAAA | TTTCCT |
| TGGGAA | TTCCCA |
| AGAGAA | TTCTCT |

The hexamers in the second list (right column) are inverted and complementary to those in the first list (left column) since hexamers from each of both lists are designed for belonging to the two amplification primers, one being the “forward” primer, thus conventionally of an identical sequence to the one of the matrix to be amplified, and the other being the reverse primer, so conventionally of an inverted and complementary sequence to the one of the matrix to be amplified. Therefore, both primers can anneal alternately on the DNA strands synthesized from the initial matrix during the PCR cycles.

The present invention also relates to a pair of primers for detecting microorganisms including a first forward primer and a second primer, wherein the first primer includes, at its 3′ end, a first hexamer as defined above and the second primer includes, at its 3′ end, a second hexamer as defined above, wherein the first and second primers may be either a forward primer and a reverse primer or a reverse primer and a forward primer.

Preferably, the first or second primer includes at its 5′end a tag selected from: FR20: 5′-GCCGGAGCTCTGCAGATATC-3′ or its variant, Fr20sb: 5′-GCCGGAGCTCTGCAGATATCAGGGCGTGGT-3′, BOP: 5′-CGGTCATGGTGGCGAATAAA-3′ or its variant, BOPsb: 5′-CGGTCATGGTGGCGAATAAATCGAGCGGC-3′, with the proviso that the first primer tag is different from the second primer tag and does not correspond neither to one of its variants.

Specifically, the first and second primers have the sequence selected from: BOPsb6.10 5′-CGGTCATGGTGGCGAATAAATCGAGCGGCTTGTAA-3′ and Fr20sb: 5′-GCCGGAGCTCTGCAGATATCAGGGCGTGGTTTACAA-3′ or FR20: 5′-GCCGGAGCTCTGCAGATATCTTACAA-3′ and BOP: 5′-CGGTCATGGTGGCGAATAAATTGTAA-3′.

The present invention also relates to a method for identifying (a) microorganism(s) in a sample containing the pair of primers as defined above, wherein said method includes the following steps consisting in:
i) if necessary, preparing said sample to remove nucleic acids which are not derived from the searched microorganism(s),
ii) if the sample obtained at step i) is a RNA, performing a step of reverse transcription with said first primer or with the second primer and an enzyme with reverse transcriptase activity,
iii) adding a PCR reaction mixture containing the first and second primers and running a first pre-amplification PCR with an annealing phase at low temperature, like around 45° C., and optionally a second PCR with an annealing phase at high temperature, like 50° C.,
iv) analysing the PCR results.

Appropriately, the products generated from step iv) are analysed on an agarose gel.

Preferably, the PCR bands obtained on said agarose gel are cut, purified and sequenced.

According to another feature of the invention, at least two of the various steps i), ii), iii) and iv) of the method are performed in the same reaction tube.

According to another feature of the invention, the first pre-amplification PCR includes at least 2 cycles.

Preferably, each cycle of the first PCR includes a denaturation phase at 90-99° C., preferably 94° C., preferably for substantially 30 s, an annealing phase at low temperature of 30-50° C., preferably 37° C. for substantially 30 s, and an elongation phase at 60-80° C. for substantially 2 min.

Appropriately, the second PCR is a conventional PCR including 35 cycles.

An aim of the present invention concerns also a kit for identifying (a) microorganism(s), characterised in that it comprises:

a—the pair of primers as defined above,

b—optionally a PCR reaction mixture,

c—and a manual of instructions.

### DETAILED DESCRIPTION OF THE INVENTION

In order to get across the idea of the subject-matter of the invention, embodiments will be described. The following description of the invention is intended as purely illustrative and non-limiting examples, with reference to the accompanying drawings:

On the drawing:

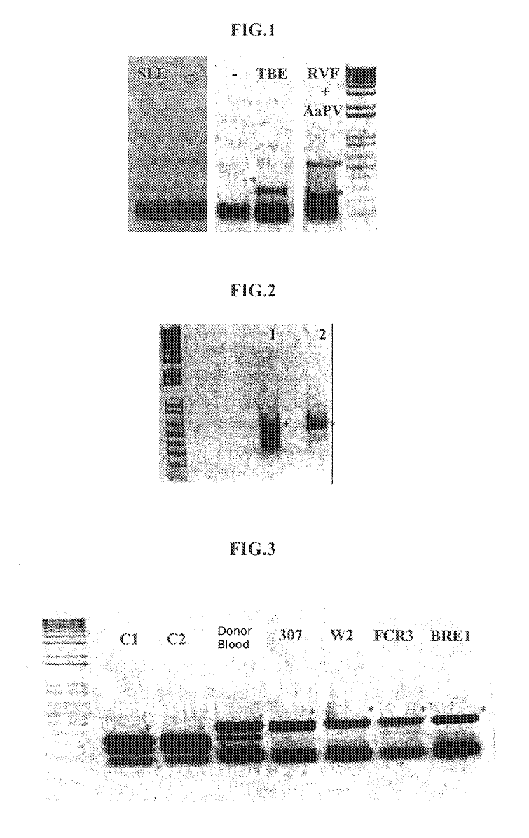

FIG. 1 shows PCR bands obtained with the method according to the invention from samples of viral culture supernatants of: St. Louis Encephalitis (SLE), Tick Borne Encephalitis (TBE) and Rift Valley Fever (RVF), and non infected cells;

FIG. 2 shows PCR bands obtained with the method according to the invention from samples derived from an nucleic acid extract of a patient blood sample (gel track 1) and from the culture supernatant of said blood sample (gel track 2);

FIG. 3 represents PCR bands obtained with the method according to the invention from samples derived from: a culture supernatant of an unknown virus derived from a small outbreak of dermatological disorders in a senior citizen home (collaboration with a virology Unit in a medical hospital, samples C1 and C2), blood from donors and parasite culture supernatants cultured with this blood (malaria, collaboration with a parasitology laboratory working on malaria, donor blood samples and malarial parasite strains 307, W2, FCR3, BRE1);

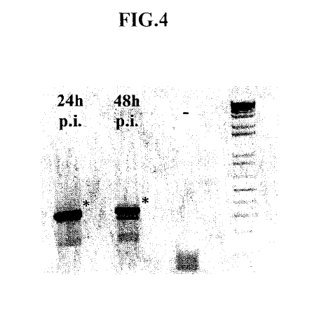

FIG. 4 represents PCR bands obtained with the method according to the invention from a viral strain sample derived from a case as diagnosed hemorrhagic Dengue 3 from Cambodia.

### A—DESIGNING THE PRIMERS

### Hybridization Sequences:

To overcome the above-mentioned disadvantages, primers whose hexamer has a fixed sequence and which anneal “frequently” have been used instead of primers with degenerate hexamers 6N which anneal everywhere, as described in Allander et al.

Mathematically, a hexamer with a given sequence anneals at least once every 4096 nucleotides (¼⁶). Biologically, all sequences of 6 nucleotides are not equivalent (typically, a series of 6 guanosines is rare).

On computer, a cutting of the genome sequences from five different viral families (measles, ebola, dengue 2 (×2), dengue 3, available on PubMed) into groups of 6 successive nucleotide has been done. Then, a classification according to their occurrence rate has been performed in order to select the most represented hexamers on average in the viral genomes. The number of selected virus (5) is low, but, in all tests performed, adding new viruses has not changed the hexamers on top of the classification.

It was not possible to find heptamers (7 nt) let alone octamers (8 nt) common between several viruses.

A certain number of hexamer sequences has been tested to retain suitable sequences, which anneal sufficiently frequently to amplify any nucleic acid but not too frequently in order not to amplify a too high number of sequences for a given nucleic acid source. A first primer (forward or reverse) is based on an hexamer, a second primer (reverse or forward, depending on the first primer) is based on a second hexamer. In particular, the pairs of hexamers that can be used are summarized in the above table. A pair of hexamers suitable for the present invention can be the pair consisting of TTGTAA as first primer and TTACAA as second primer, for example.

### Tag Sequences:

In the technique used by Allander, only one tag is used for PCR. The amplified PCR fragments have therefore the same ends (the tag and its complementary tag). This end symmetry prevents a direct sequencing of the PCR products (there would be a concomitant sequencing of the fragment in both ways, so it would not be possible to solve the double sequence).

Two different tags have been selected: FR20: 5′-GCCGGAGCTCTGCAGATATC-3′ or its variant, Fr20sb: 5′-GCCGGAGCTCTGCAGATATCAGGGCGTGGT-3′, BOP: 5′-CGGTCATGGTGGCGAATAAA-3′ or its variant, BOPsb: 5′-CGGTCATGGTGGCGAATAAATCGAGCGGC-3′.

Those primers allow the amplification of “asymmetrical” PCR fragments likely to be sequenced directly. The use of different and non-complementary tags between both primers allows indeed the direct sequencing of the PCR fragment without having a PCR amplicon that folds over itself (hard to amplify). Therefore, symmetrical amplicons are eliminated.

For the following examples, the pair of primer used is the following: Fr20sb: 5′-GCCGGAGCTCTGCAGATATCAGGGCGTGGTTTACAA-3′ (first primer) and BOPsb6.10 5′-CGGTCATGGTGGCGAATAAATCGAGCGGCTTGTAA-3′ (second primer).

### B—PROCEDURE FOR THE METHOD ACCORDING TO THE INVENTION

### Also Referred as “RT-PCR or Random PCR Method”

Similarly to Allander et al., as the technique employed amplifies any nucleic acid it is convenient to use as starting material a sample containing only the studied microorganism. Infected cells are typically a bad starting material as the host ribosomal RNAs form a great majority, however the clarified supernatant of those same non lysed cells is a suitable sample. This specific sample type will be illustrated in the examples.

The purified nucleic acid is subjected to a first step of reverse transcription with the first primer and an enzyme having a reverse transcription activity, such as AMV RT, Promega, etc. (enzymes known by the skilled person). This step lasts approximately 40 minutes and allows the synthesis of a first DNA strand when the starting sample is RNA. If the starting sample contains only DNA, this step has no effect.

A PCR reaction mixture, known from the skilled person (type Master Mix, Qiagen) and containing the second primer, is added into the same tube. The whole tube is subjected to 5 poorly selective PCR cycles (denaturation at 94° C., 30 sec, low temperature hybridization at 37° C., 30 sec, elongation at 72° C., 2 minutes). This step allows the synthesis of the second strand starting from a RNA sample, or of the first then second strand starting from a DNA sample. This step lasts approximately 40 minutes.

The two primers are once again added into the same tube in order to perform the amplification by 35 conventional PCR cycles. This step lasts approximately 2.5 hours.

In an alternative embodiment, the two foregoing PCR steps can be joined in a single step if the user decided to perform the two PCR phases of 5 cycles, followed by 35 cycles one directly after the other in the thermocycler, without any additional intermediate addition of the two primers, said primers being provided in excess at the beginning of the reaction.

The post-PCR samples are then analyzed on an agarose gel and the generated PCR bands, if any, are cut, purified and sequenced. Approximately 80% of the bands directly sequenced result in achieving a sequence.

It is worth noting that, on viral culture supernatants, the step of nucleic acid extraction is not critical: a few microliters of culture directly added to the RT mix are sufficient to perform the reaction.

Thus, the reaction designed by the present applicant allows the amplification, in a single reaction tube, using common laboratory protocols and materials, in less than 4 hours, of at least one PCR or RT-PCR band starting from any nucleic acid of approximately 3000 nucleotides or more, this band being directly sequencable in a great majority of cases.

Some examples of identification according to the invention, which are purely illustrative and do not limit the scope of the invention reach will now be described.

### EXAMPLE 1

### Parvovirus and C6/36

The method for identifying a microorganism has been validated on viruses kept in the laboratory and researched blindly.

The method according to the invention referred to in point B has been carried out by a laboratory technician on viral culture supernatants of St. Louis encephalitis (SLE), Tick Borne Encephalitis (TBE) and Rift Valley fever (RVF), and non infected cells (see FIG. 1). Asterisks show the sequenced bands.

As shown in FIG. 1, amplification bands have indeed been found, which corresponded, after sequencing, to the expected viruses in each case.

This study further allowed us to amplify 2 PCR bands in the sample RVF, one corresponding actually to the expected RVF virus, the other corresponding to an insect parvovirus, Aedes Alpopictus Parvovirus (AaPV). After some bibliographical research, it was found to be a parvovirus previously found as chronically infecting a C6/36 cell line which cells derive from Aedes albopictus (Boublik Y. et al, Cloning, sequencing and infectious plasmid construction of a new parvovirus, the Aedes Albopictus Parvovirus, pathogenic for the mosquito Aedes Aegypti larvae, doctoral thesis from Aix Marseille University, 1993).

### EXAMPLE 2

### Patient X Blood

A blood sample whose diagnosis was initially not very clear has been studied using the present method.

This blood contained a virus that has been previously amplified by culturing.

The random RT-PCR protocol according to the invention has been used on two samples: one nucleic acid extract obtained from a patient blood sample (gel track 1) and the culture supernatant (gel track 2).

As shown in FIG. 2, the blood sample (1) led to the identification of the patient ribosomal RNAs which were in the extract. This result only confirmed that the patient was indeed a homo sapiens sapiens. However, it points out the importance of the sample preparation before the random PCR in order to remove the nucleic acids which do not originate from the researched microorganism.

The viral culture supernatant (gel track 2) allowed the identification of the dengue 2 virus from a Martinican strain. This result confirmed the one the diagnosing team had found meanwhile.

### EXAMPLE 3

### Reproducibility and Non Viral Microorganisms (FIG. 3)

The unique identification method according to the invention was carried out on various samples (see FIG. 3).

All the sequenced bands corresponded to mycoplasms having contaminated the cultures, of two different strains, one for samples C1 and C2 from an hospital laboratory, the second one for the malarial parasite culture samples.

This series of experiments also allows validating the reproducibility of the method according to the invention. Indeed, the reaction principle uses in fact more a probability than a chance: if, for an unknown sample, the employed primers have a certain probability to anneal somewhere on the nucleic acid of interest, for a given sample, the primers always anneal to the same sequence. In other words, the same causes result in the same effects: samples containing the same microorganism lead to an amplification of the same sequences, reproducibly, thus to the generation of the same PCR or RT-PCR bands.

Therefore, mycoplasms of different strains, coming from the hospital laboratory or from donor blood, lead to the generation of different PCR bands, but samples from similar origin, on one hand samples from the hospital laboratory, on the other hand from a culture of the same blood, individually lead to the generation of the same PCR bands leading to the same sequences in each of both considered groups.

This represents the clear advantage over the method of Allander et al.: on the series presenting a similar origin, it is not required to sequence all the identical bands because they will certainly lead to the same final sequences.

### EXAMPLE 4

### Unknown Viruses

A viral strain coming from a case diagnosed as hemorrhagic Dengue 3 fever from Cambodia was then analysed.

It appeared quickly that the behaviour of the cell cultured virus was surprising for a dengue (cytopathogenic effect particularly strong and quickly developed).

Taqman RT-PCR assays specific of the hemorrhagic Dengue 3 fever were found negative, as were the other dengue serotypes or even the universal dengue assays which were performed.

The random RT-PCR method was applied: to two supernatant extracts of a culture, on VERO cells, of this virus (24 h post infection=24 h p.i. and 48 h post infection=48 h p.i.) and to a control on a supernatant of non infected cells (−) (FIG. 4).

The generated nucleic acid sequences, identical, do not correspond to any available sequence in the databases (PubMed). However, the translated sequence presents homologies with the sequence of a polymerase from a bunyaviridae, CiLV or Citrus Leprosis virus, a lemon tree arbovirus transmitted by a mite.

This result may seem surprising: an homology with a plant virus for a human haemorrhage. However, if the bunyaviridae family comprises mammal viruses (bunyavirus, nairovirus, hantavirus), it also comprises a whole plant virus genus, the Tospovirus.

The random RT-PCR thus gives a clue for looking for a virus of the bunyaviridae family.

The method according to the invention gives a very quick result when the generated sequence finds a significant homology in the databases. However, given that this generated sequence is not selected, it could correspond to genome unsequenced regions or to unknown microorganisms. In that case, it serves as a clue for directing the identification by other means that will require more time.

Therefore, the method according to the invention is easy to implement in a common laboratory, it is fast, it allows performing controls in the course of the reaction so as to avoid unnecessary blind sequencing. Moreover, on the various examples showed therein, the method according to the invention demonstrated its efficiency in terms of molecular biology and its ability to provide information on studied pathogens.

Furthermore, the random RT-PCR method can be applied to search for any microorganism, from any species, as long as it possesses a nucleic acid.

Even though the invention was described in relation to a specific embodiment, it is obviously not limited thereto and it comprises obviously all the technical equivalents to the means described therein, the combination thereof, provided they are within the scope of the invention.

1-13. (canceled)

14. A pair of hexamers for PCR for detecting microorganisms, comprising a first hexamer to be positioned at the 3′ end of a first primer and a second hexamer to be positioned at the 3′ end of a second primer, said pair of hexamers being obtainable by the method consisting in:
a) cleaving the genome sequences of at least five viruses from different families into groups of six successive nucleotides,
b) classifying the sequences of six nucleotides, referred as hexamers, according to their occurrence rate,
c) selecting the pairs of hexamers having the most represented occurrence rate, like the first twenty hexamers, preferably the first ten, more preferably, the first five having the higher occurrence rate when compared to the other pairs of hexamers, in order to obtain a pair of primers able to anneal statistically frequently on any nucleic acid matrix.

15. A pair of hexamers according to claim 14, wherein the first and second hexamers are selected from:

| First hexamer | Second hexamer |
|---|---|
| (5′ to 3′ reading) | (5′ to 3′ reading) |
| TTGTAA | TTACAA |
| TCATCA | TTGTAA |
| AAAGAA | TTGTAA |
| GGAAAA | TTTTCC |
| GAAAGA | TCTTTC |
| GGAAGA | TCTTCC |
| AGAAAA | TTTTCT |
| GGGAAA | TTTCCC |
| AAGAAA | TTTCTT |
| AACATG | CATGTT |
| GAAAAA | TTTTTC |
| AAGGAA | TTCCTT |
| GGAAAG | CTTTCC |
| TGGAAA | TTTCCA |
| AAAAAA | TTTTTT |
| GAAGAA | TTCTTC |
| AGGAGA | TCTCCT |
| AAAAAG | CTTTTT |
| AAAAGA | TCTTTT |
| ACATGG | CCATGT |
| AGAAGA | TCTTCT |
| TGATGA | TCATCA |
| TGGAAG | CTTCCA |
| CATGGA | TCCATG |
| AGGAAA | TTTCCT |
| TGGGAA | TTCCCA |
| AGAGAA | TTCTCT |

16. A pair of primers for detecting microorganisms, including a first forward primer and a second primer, wherein the first primer includes, at its 3′ end, a first hexamer as defined above and the second primer includes, at its 3′ end, a second hexamer as defined in claim 14, wherein the first and second primers may be either a forward primer and a reverse primer or a reverse primer and a forward primer.

17. A pair of hexamers according to claim 16, wherein the first or second primer includes at its 5′ end a tag selected from: FR20: 5′-GCCGGAGCTCTGCAGATATC-3′ (SEQ ID NO: 1) or its variant, Fr20sb: 5′-GCCGGAGCTCTGCAGATATCAGGGCGTGGT-3′ (SEQ ID NO: 2), BOP: 5′-CGGTCATGGTGGCGAATAAA-3′ (SEQ ID NO: 3) or its variant, BOPsb: 5′-CGGTCATGGTGGCGAATAAATCGAGCGGC-3′ (SEQ ID NO: 4), with the proviso that the first primer tag is different from the second primer tag and does not correspond neither to one of its variants.

18. A pair of hexamers according to claim 16, wherein the first and second primers have the sequence selected from: BOPsb6.10 5′-CGGTCATGGTGGCGAATAAATCGAGCGGCTTGTAA-3′ (SEQ ID NO: 5) and Fr20sb: 5′-GCCGGAGCTCTGCAGATATCAGGGCGTGGTTTACAA-3′ (SEQ ID NO: 6) or FR20: 5′-GCCGGAGCTCTGCAGATATCTTACAA-3′ (SEQ ID NO: 7) et BOP: 5′-CGGTCATGGTGGCGAATAAATTGTAA-3′ (SEQ ID NO: 8).

19. A method for identifying (a) microorganism(s) in a sample containing the pair of primers as defined in claim 16, wherein said method includes the following steps consisting in:
i) if necessary, preparing said sample to remove nucleic acids which are not derived from the searched microorganism(s),
ii) if the sample obtained at step i) is a RNA, performing a step of reverse transcription with said first primer or with the second primer and an enzyme with reverse transcriptase activity,
iii) adding a PCR reaction mixture containing the first and second primers and running a first pre-amplification PCR with an annealing phase at low temperature, like around 45° C., and optionally a second PCR with an annealing phase at high temperature, like 50° C.,
iv) analysing the PCR results.

20. A method for identifying (a) microorganism(s) according to claim 19, wherein the products generated from step iv) are analysed on an agarose gel.

21. A method for identifying (a) microorganism(s) according to claim 20, wherein the PCR bands obtained on said agarose gel are cut, purified and sequenced.

22. A method for identifying (a) microorganism(s) according to claim 19, wherein at least two of the various steps i), ii), iii) and iv) of the method are performed in the same reaction tube.

23. A method for identifying (a) microorganism(s) according to claim 19, wherein the first pre-amplification PCR includes at least 2 cycles.

24. A method for identifying (a) microorganism(s) according to claim 23, wherein each cycle of the first PCR includes a denaturation phase at 90-99° C., preferably 94° C., preferably for substantially 30 s, an annealing phase at low temperature of 30-50° C., preferably 37° C. for substantially 30 s, and an elongation phase at 60-80° C. for substantially 2 min.

25. A method for identifying (a) microorganism(s) according to claim 19, wherein the second PCR is a conventional PCR including 35 cycles.

26. A kit for identifying (a) microorganism(s), characterised in that it comprises:
a—the pair of primers as defined in claim 19,
b—optionally a PCR reaction mixture,
c—and a manual of instructions.


SEQUENCE LISTING (8 sequences)

SEQ ID NO: 1 - DNA, 20 bp, Artificial Sequence
  Tag to be placed at the 5′ extremity of a PCR primer
gccggagctctgcagatatc

SEQ ID NO: 2 - DNA, 30 bp, Artificial Sequence
  Tag to be placed at the 5′ extremity of a PCR primer
gccggagctctgcagatatcagggcgtggt

SEQ ID NO: 3 - DNA, 20 bp, Artificial Sequence
  Tag to be placed at the 5′ extremity of a PCR primer
cggtcatggtggcgaataaa

SEQ ID NO: 4 - DNA, 29 bp, Artificial Sequence
  Tag to be placed at the 5′ extremity of a PCR primer
cggtcatggtggcgaataaatcgagcggc

SEQ ID NO: 5 - DNA, 35 bp, Artificial Sequence
  PCR primer
cggtcatggtggcgaataaatcgagcggcttgtaa

SEQ ID NO: 6 - DNA, 36 bp, Artificial Sequence
  PCR primer
gccggagctctgcagatatcagggcgtggtttacaa

SEQ ID NO: 7 - DNA, 26 bp, Artificial Sequence
  PCR primer
gccggagctctgcagatatcttacaa

SEQ ID NO: 8 - DNA, 26 bp, Artificial Sequence
  PCR primer
cggtcatggtggcgaataaattgtaa